(19) United States
(12) Patent Application Publication
(10) Pub. No.: US 2025/0152514 A1
(43) Pub. Date: May 15, 2025

(54) MICROPARTICLES HAVING ZWITTERIONICALLY MODIFIED SURFACE, DRUG DELIVERY SYSTEM COMPRISING THE SAME, AND A PROCESS FOR PREPARING THE SAME

(71) Applicant: UIF (UNIVERSITY INDUSTRY FOUNDATION), YONSEI UNIVERSITY, Seoul (KR)

(72) Inventors: Jinkee HONG, Seoul (KR); Sungwon JUNG, Seoul (KR)

(73) Assignee: UIF (UNIVERSITY INDUSTRY FOUNDATION), YONSEI UNIVERSITY, Seoul (KR)

(21) Appl. No.: 18/937,121

(22) Filed: Nov. 5, 2024

(30) Foreign Application Priority Data
Nov. 9, 2023 (KR) 10-2023-0154104

(51) Int. Cl. A61K 9/16; A61K 9/00; A61K 9/50; A61K 31/4418; A61K 31/496; A61K 31/573

(52) U.S. Cl. CPC A61K 9/1647; A61K 9/0078; A61K 9/1694; A61K 9/5015; A61K 31/4418; A61K 31/496; A61K 31/573

(57) ABSTRACT

The present invention relates to microparticles, drug delivery systems, and methods of preparing the same, wherein the surface of poly(lactic-co-glycolic acid) (PLGA) is modified with amphoteric ions, Due to the water film formed by the modification of the amphoteric ions, the delivery of microparticles (or drug delivery systems) to the lungs through nebulizing can reach the alveoli with high delivery efficiency without being removed by the mucin mucus layer present in the airway.

### CROSS-REFERENCE TO RELATED APPLICATIONS

This application claims priority under 35 U.S.C. § 119 to Korean Patent Application No. 10-2023-0154104, filed on Nov. 9, 2023 in the Korean Intellectual Property Office, the disclosure of which is incorporated herein by reference in its entirety.

### TECHNICAL FIELD

The present invention relates to microparticles, drug delivery systems and methods of preparation thereof that, when delivered to the lungs, can reach the alveoli with high delivery efficiency without being removed by the mucin mucus layer present in the airways.

### BACKGROUND OF THE INVENTION

Fibrosis is the thickening and scarring of connective tissue due to injury, characterized by excessive proliferation of fibroblasts and accumulation of extracellular matrix (ECM) components. It is commonly observed in organs including the lungs, liver, and kidneys, and can lead to the destruction of tissue structure and severe impairment of organ function. In fact, fibrosis can occur in almost any organ and is a major cause of end-organ failure and death in a variety of chronic diseases.

A common feature of pulmonary fibrosis is the excessive proliferation of fibroblasts around the air sacs (alveoli) of the lungs. Extensive biomedical research has shown that increased fibroblast numbers and excessive ECM deposition in the lungs will eventually lead to destruction of alveolar structure, decreased lung compliance, and impaired gas exchange function.

The most common type of pulmonary fibrosis is idiopathic pulmonary fibrosis (IPF). This disorder eventually affects the entire lung lobe, but it starts with small fibrotic lesions that develop in the circumferential area and slowly progress inward, and this fibrosis can eventually cause respiratory failure. As mentioned above, IPF is a fatal disease and the median survival time from diagnosis is only 2 to 4 years. Scientifically speaking, the mechanism and nature of the pathological progression of IPF are not yet fully understood, but many studies have shown that type II alveolar cells (AT2), derived from a specific subgroup of alveolar epithelial cells, play a role in the pathogenesis of IPF.

In patients with pulmonary fibrosis, lung compliance decreases and gas exchange is disrupted, ultimately leading to respiratory failure and death. In the United States, it is estimated that 1 in 200 adults over the age of 65 has IPF, with a median survival time of 2 to 4 years. In China, the incidence of IPF is estimated to be 3-5/100,000, accounting for about 65% of all interstitial lung diseases. Typically, pulmonary fibrosis is diagnosed between the ages of 50 and 70, with a male-to-female ratio of 1.5 to 2:1, and patients typically have a survival time of only 2 to 5 years.

On the other hand, safe and efficient drug delivery technology for drug therapy has been studied for a long time, and various delivery vehicles and delivery technologies have been developed. Delivery systems are broadly categorized into viral delivery systems, such as adenoviruses and retroviruses, and non-viral delivery systems, such as cationic lipids and cationic polymers. Viral delivery systems are exposed to risks such as non-specific immune responses and are known to have many problems in commercialization due to complex production processes. Therefore, recent research has been directed toward improving their shortcomings by using non-viral delivery systems.

Compared to viral vectors, non-viral vectors have the advantage of fewer side effects in terms of in vivo safety and lower production costs in terms of economics.

Therefore, there is a need for a non-viral drug delivery system that can reach the alveoli with high delivery efficiency without being removed by the mucin mucus layer present in the airways upon delivery to the lungs.

### SUMMARY OF THE INVENTION

### Problem to be Solved

The object of the present invention is to provide amphoteric surface-modified microparticles that, when delivered to the lungs, are not removed by the mucin mucus layer present in the airways and can reach the alveoli with high delivery efficiency.

It is another object of the present invention to provide drug delivery systems comprising microparticles having a surface modified with said amphoteric ions.

It is another object of the present invention to provide a method for preparing microparticles having a surface modified with said amphoteric ions.

It is another object of the present invention to provide a method for preparing a drug delivery system having a surface modified with an amphoteric ion and containing two drugs.

It is another object of the present invention to provide pharmaceutical compositions for the treatment of pulmonary diseases comprising said drug delivery systems as active ingredients.

### Solution to the Challenge

The microparticles of the present invention for achieving the aforementioned objectives may have a surface of poly(lactic-co-glycolic acid) (PLGA) modified with amphoteric ions.

The average diameter of said microparticles may be from 1 to 4 micrometers.

The zwitterion may be formed using sulfobetaine methacrylate (SBMA), 2-methacryloyloxyethyl phosphorylcholine (MPC), or carboxybetaine methacrylate (CBMA).

Further, the drug delivery system of the present invention for accomplishing the other purposes described above may be a poly(lactic-co-glycolic acid) (PLGA) surface modified with an amphoteric ion and loaded with a drug.

The target site of said drug delivery system may be the lungs, in particular the alveoli.

The drug delivery system may be administered intranasally or endobronchially to reach the lungs, in particular the alveoli.

The drug delivery system may be both hydrophobic and hydrophilic drug-loaded.

The hydrophobic drug may be pirfenidone or nintedanib; the hydrophilic drug may be dexamethasone, prednisone (PD), prednisolone (PDS), or methylprednisolone (MP).

Further, a method of preparing the microparticles of the present invention to accomplish another object of the present invention comprises (A) performing an aminolysis reaction to form an amine group on a surface of a poly(lactic-co-glycolic acid) (PLGA) particle; (B) reacting the PLGA particles having amine groups formed on said surface with an initiator of an atomized radical polymerization (ATRP) reaction to form halogen groups; and (C) reacting said halogen groups with an amphoteric compound to obtain PLGA micro-particles having amphoteric ions modified on the surface.

In step (A) above, the PLGA particles may be prepared by (a) dissolving PLGA in an organic solvent, adding distilled water, and then treating with an ultrasonicator to prepare an emulsion; (b) dropping said emulsion into polyvinyl alcohol to form a double emulsion; and (c) stirring said double emulsion to obtain solid phase PLGA particles.

The amphoteric compound in step (C) above may be sulfobetaine methacrylate (SBMA), 2-methacryloyloxyethyl phosphorylcholine (MPC), or carboxybetaine methacrylate (CBMA). Furthermore, a method of preparing a drug delivery system of the present invention to accomplish another of the aforementioned objectives comprises (A′) performing an aminolysis reaction to form amine groups on the surface of poly(lactic-co-glycolic acid, PLGA) particles loaded with two types of drugs; (B′) reacting the PLGA particles having amine groups on said surface with an initiator of an atomization-type radical polymerization (ATRP) reaction to form halogen groups; and (C′) reacting said halogen groups with an amphoteric compound to obtain PLGA micro-particles having amphoteric ions modified on the surface.

The PLGA particles loaded with two kinds of drugs in step (A′) above, wherein the PLGA particles are prepared by (a′) dissolving the PLGA in an organic solvent in which the hydrophobic drug is dissolved, adding distilled water in which the hydrophilic drug is dissolved, and then treating the PLGA with an ultrasonicator to prepare an emulsion; (b′) dropping said emulsion into polyvinyl alcohol to form a double emulsion; and (c′) stirring said double emulsion to obtain PLGA particles that are solid and contain two kinds of drugs.

The hydrophobic drug may be pirfenidone or nintedanib; the hydrophilic drug may be dexamethasone, prednisone (PD), prednisolone (PDS), or methylprednisolone (MP).

The amphoteric compound in step (C′) may be sulfobetaine methacrylate (SBMA), 2-methacryloyloxyethyl phosphorylcholine (MPC), or carboxybetaine methacrylate (CBMA). Furthermore, pharmaceutical compositions for the treatment of pulmonary diseases of the present invention to accomplish another of the aforementioned objectives may comprise said drug delivery system as an active ingredient.

The pharmaceutical composition may be for intranasal administration or for endobronchial administration.

Said lung disease may be one or more selected from the group consisting of idiopathic pulmonary fibrosis (IPF), acute lung injury, bleomycin-induced pulmonary fibrosis, mechanical respiratory induced lung injury, chronic obstructive pulmonary disease (COPD), chronic bronchitis, and emphysema.

### Effect of Invention

The pulmonary delivery construct of the present invention is a poly(lactic-co-glycolic acid) (PLGA) surface modified with an amphoteric ion or a poly(lactic-co-glycolic acid) (PLGA) surface modified with an amphoteric ion that is loaded with two or more drugs, The water film formed by the amphoteric modification enables the delivery of microparticles (or drug delivery systems) to the lungs by nebulizing to reach the alveoli with high delivery efficiency without being removed by the mucin mucus layer present in the airways.

### BRIEF DESCRIPTION OF THE DRAWINGS

FIG. 1 is a schematic diagram illustrating the process of modifying zwitterions on the surface of PLGA particles via an ATRP reaction in accordance with one embodiment of the present invention.

FIG. 2A is a scanning electron microscope (SEM) image of PLGA particles as a function of the reaction time of the aminolysis reaction; FIG. 2B is a graph showing the zeta potential results of the PLGA particles functionalized with the amine groups of FIG. 2A.

FIG. 3A is a graph of Fourier transform infrared (FTIR) spectroscopy measurements of PLGA particles after the aminolysis reaction; FIG. 3B is a graph of the results of TNBS analysis to quantify the amine groups formed on the surface of PLGA particles after the aminolysis reaction.

FIG. 4A is a graph showing C atom results from X-ray photoelectron spectroscopy (XPS) of PLGA particles at each stage according to one embodiment of the present invention; FIG. 4B is a graph showing Br atom results from X-ray photoelectron spectroscopy (XPS) of PLGA particles at each stage according to one embodiment of the present invention.

FIG. 5 shows transmission electron microscopy (TEM) measurements of PLGA particles, SBPG particles, and MPG particles.

FIG. 6A is a graph of laser particle size analysis of PLGA particles, SBPG particles, and MPG particles; FIG. 6B is a graph of zeta potential results of PLGA particles, NH₂-PG particles, Br-PG particles, SBPG particles, and MPG particles.

FIG. 7A is a graph of the results of X-ray photoelectron spectroscopy for PLGA particles, NH₂-PG particles, Br-PG particles, SBPG particles, and MPG particles; FIGS. 7B through 7D are graphs of the results of X-ray photoelectron spectroscopy for Br-PG particles, SBPG particles, and MPG particles, respectively.

FIG. 8A is a photograph of EDS analysis of PLGA particles, SBPG particles, and MPG particles; and FIG. 8B is a graph quantifying the results of the EDS analysis of FIG. 8A.

FIG. 9 is an experimental design to analyze particle permeability in mucin mucus for PLGA particles, SBPG particles, and MPG particles.

FIGS. 10A to 10C are graphs analyzing particle permeability in mucin mucus for PLGA particles, SBPG particles, and MPG particles, respectively.

FIG. 11A is a graph quantifying the mucin protein adsorbed on PLGA particles, SBPG particles, and MPG particles; FIG. 11B is a graph quantifying the particles that penetrated the mucin layer on PLGA particles, SBPG particles, and MPG particles using transwells.

FIG. 12A is an SEM image for stability analysis of PLGA particles, SBPG particles, and MPG particles after nebulizing with a nebulizer; FIG. 12B is a graph showing the zeta potential of each particle after nebulizing with a nebulizer.

FIG. 13 is a graph showing the cytotoxicity of SBPG particles and MPG particles against lung tissue-derived cells.

FIG. 14A is a confocal microscopy image measured after rhodamine (RhB) and methylene blue (MB) were immersed in PLGA particles; FIG. 14B is a graph of UV-vis measurements of a zwitterionic surface-modified drug delivery system (DPPG) loaded with Dex and Pir drugs; and FIG. 14C is a graph quantifying the measurements in FIG. 14B.

### SPECIFIC DETAILS FOR PRACTICING THE INVENTION

The present invention relates to microparticles, drug delivery systems and methods of preparation thereof that, when delivered to the lungs, can reach the alveoli with high delivery efficiency without being removed by the mucin mucus layer present in the airways.

The microparticles or drug delivery systems surface modified with the amphoteric ions of the present invention can be efficiently delivered within the lung environment without being removed by the mucin mucus layer present in the airways and elsewhere, targeting the alveoli in particular. Specifically, the amphoteric ions formed on the surface of the amphoteric ion-functionalized microparticles or drug delivery systems form a water film, which can block the adsorption of mucin as well as other proteins, and the water film of the amphoteric ion-functionalized microparticles or drug delivery systems can reduce the interaction with mucin proteins, thereby exhibiting high anti-mucin ability and high stability in mucin mucus.

The present invention will now be described in detail.

The amphoteric surface-modified microparticles of the present invention are poly(lactic-co-glycolic acid, PLGA) microparticles whose surface is amphoterically modified and have an average diameter of 1 to 4 m, preferably 2.0 to 3.5 m, a size suitable for reaching the alveoli. The amphoteric compounds that are modified on the surface of the microparticles whose surfaces are modified with the amphoteric ions of the present invention include, but are not particularly limited to, any substance capable of forming amphoteric ions. Preferred examples are sulfobetaine methacrylate (SBMA), 2-methacryloyloxyethyl phosphorylcholine (MPC) or carboxybetaine methacrylate (CBMA).

When the surface of the microparticles modified with amphoteric ions is modified, a water film is formed, and during pulmonary delivery by inhalation (e.g., nebulizing), the pulmonary delivery structure can be delivered to the alveoli inside the lungs without being removed by the mucin mucus layer present in the airway due to the water film formed.

Specifically, the aqueous film formed by the amphoteric modification prevents the amphoteric surface-modified microparticles from adsorbing to the mucin membrane and increases the permeability of the mucin membrane, allowing efficient delivery to the alveoli.

The present invention also provides a drug delivery system comprising microparticles having a surface modified with said amphoteric ion.

The drug delivery system of the present invention, wherein said drug delivery system is loaded with both a hydrophobic drug and a hydrophilic drug, wherein said hydrophobic drug and hydrophilic drug are released upon reaching the interior of the lungs due to the biodegradability of the zwitterionic surface-modified microparticles, and wherein said hydrophobic drug is released upon degradation of said zwitterionic surface-modified microparticles, thereby enabling the drug to reach the interior of the lungs.

The hydrophobic drug and the hydrophilic drug are not particularly limited as long as they can treat lung disease, and specifically include pirfenidone or nintedanib as hydrophobic drugs; a hydrophilic agent, such as dexamethasone, prednisone (PD), prednisolone (PDS), or methylprednisolone (MP).

The present invention can also provide a method for preparing microparticles having a surface modified with amphoteric ions.

A method for preparing a surface-modified microparticle with an amphoteric ion of the present invention, comprising (A) performing an aminolysis reaction to form an amine group on a surface of a poly(lactic-co-glycolic acid) (PLGA) particle; (B) reacting the PLGA micro-particles having amine groups formed on said surface with an initiator of an atomized radical polymerization (ATRP) reaction to form halogen groups; and (C) reacting said halogen groups with an amphoteric compound to obtain micro-particles having amphoteric ions modified on the surface.

The present invention uses an atom transfer radical addition (ATRP) reaction to modify amphoteric ions on the surface by controlling the solvent use, since the surface modification is difficult to control in organic solvents due to the characteristics of PLGA particles.

Specifically, the method for preparing the amphoteric surface-modified microparticles of the present invention comprises: performing an ester aminolysis reaction to modify an amine group on the surface of the PLGA microparticles to form an amine group (step (A)); reacting the formed amine group with an ATRP reaction initiator to form a halogen group (step (B)); and finally, reacting the formed halogen group with the amphoteric compound and the ATRP reaction initiator to form a halogen group (step (C)). Finally, the ATRP reaction of the amphoteric compound with the halogen groups formed on the surface of the PLGA micro-particles is performed to obtain micro-particles with a surface modified with amphoteric ions (step (C)).

The PLGA particles used to prepare the microparticles having a surface modified with amphoteric ions in step (A) above are prepared by (a) dissolving PLGA in an organic solvent, adding distilled water, and then treating with an ultrasonicator to prepare an emulsion (W1/O); (b) dropping said emulsion into polyvinyl alcohol to form a double emulsion (W1/O/W2); and (c) stirring said double emulsion to obtain solid phase PLGA particles.

The present invention also provides a method for preparing A drug delivery system that is loaded with two types of drugs and has a surface modified with amphoteric ions.

The manufacturing process is the same as for the zwitterionic surface-modified micro-particles above, except that the method for preparing the PLGA particles in step (A) above is different. A method for preparing A drug delivery system carrying two types of drugs and having a surface modified with an amphoteric ion of the present invention, comprising (A′) performing an aminolysis reaction to form amine groups on the surface of poly(lactic-co-glycolic acid, PLGA) particles carrying two types of drugs; (B′) reacting the PLGA particles having amine groups formed on said surface with an initiator of an atomization-type radical polymerization (ATRP) reaction to form a halogen group; and (C′) reacting said halogen group with an amphoteric compound to obtain a drug delivery system having a surface modified with an amphoteric ion. The PLGA particles used in step (A′) above to prepare a drug delivery system containing two kinds of drugs and having a surface modified with amphoteric ions, wherein the PLGA particles are prepared by (a′) dissolving the PLGA in an organic solvent in which a hydrophobic drug is dissolved, adding distilled water in which a hydrophilic drug is dissolved, and then treating the PLGA with an ultrasonicator to prepare an emulsion; (b′) dropping said emulsion into polyvinyl alcohol to form a double emulsion; and (c′) stirring said double emulsion to obtain PLGA particles that are solid and carry two kinds of drugs.

In other words, when manufacturing the PLGA particles used in step (A′), an organic solvent containing a hydrophobic drug and distilled water containing a hydrophilic drug are added sequentially to make an emulsion, and then a double emulsion is obtained by utilizing the emulsion, so that different types of drugs can be immersed. In this way, PLGA particles containing different types of drugs can be modified with amphoteric ions on the surface according to the method of the present invention to obtain a drug delivery system that can be delivered to the alveoli.

The present invention may also provide pharmaceutical compositions for the treatment of lung disease comprising said drug delivery system as an active ingredient.

The pharmaceutical composition may be used for intranasal or endobronchial administration, and is indicated for the treatment of lung diseases, including idiopathic pulmonary fibrosis (IPF), acute lung injury, and bleomycin-induced pulmonary fibrosis, mechanical respiratory induced lung injury, chronic obstructive pulmonary disease (COPD), chronic bronchitis, and emphysema, and at least one selected from the group consisting of.

The present invention also provides a novel use for a drug delivery system in which the surface of poly(lactic-co-glycolic acid) (PLGA) is modified with zwitterionic ions for the manufacture of a pharmaceutical for the treatment of lung disease.

In addition to the drug delivery system having as an active ingredient a poly(lactic-co-glycolic acid, PLGA) surface modified with an amphoteric ion, the “pharmaceutical composition” may further comprise suitable carriers, excipients and diluents conventionally used in the preparation of pharmaceutical compositions and the like.

A “carrier” is a compound that facilitates the incorporation of a compound into a cell or tissue. A “diluent” is a compound that stabilizes the biologically active form of the target compound, as well as dilutes the compound in water, which makes the compound soluble.

Said carriers, excipients and diluents need not be particularly limited to, but include, for example, lactose, glucose, sugar, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils.

The dosage of the pharmaceutical composition may vary depending on the age, sex, and weight of the patient or treated animal and will depend on, among other things, the condition of the individual being treated, the specific category or type of disease being treated, the route of administration, and the properties of the therapeutic agent being used.

The pharmaceutical compositions may be used individually as therapeutic agents or in combination with other therapeutic agents, and may be administered sequentially or concurrently with conventional therapeutic agents.

Below, preferred embodiments of the present invention are described for the purpose of illustrating the invention, but it will be apparent to those skilled in the art that various changes and modifications are possible within the scope of the invention and the technical idea, and that such changes and modifications fall within the scope of the appended patent claims.

### Examples and Experimental Examples Regarding SBPG Particles and MPG Particles

### Preparatory Example 1. PLGA Particles

PLGA particles are prepared by a double emulsion method, targeting a size of 2-3 m for pulmonary delivery.

After 75 mg of PLGA was dissolved in 3 mL of dichloromethane, 1 mL of tertiary distilled water (deionized water, DW) was added, and an emulsion (W1/O) was prepared using an ultrasonicator (VC-505, SONICS). The emulsion prepared above was added 4 ml to a polyvinyl alcohol (PVA) solution (2 wt % PVA dissolved in 100 wt % total) under stirring to form a double emulsion. Stirring was then continued for 6 hours to evaporate the remaining dichloromethane in the solution to form solidified PLGA particles. Finally, the solution and particles were separated using a centrifuge (13000 rpm, 10 min, VARISPIN15R, Hanil Scimed), washed three times with DW, and lyophilized (FreeZone 2.5 Liter, −50° C., LABCONCO) to obtain PLGA particles of 2 μm (average diameter).

### Examples 1 and 2. SBPG and MPG Particles

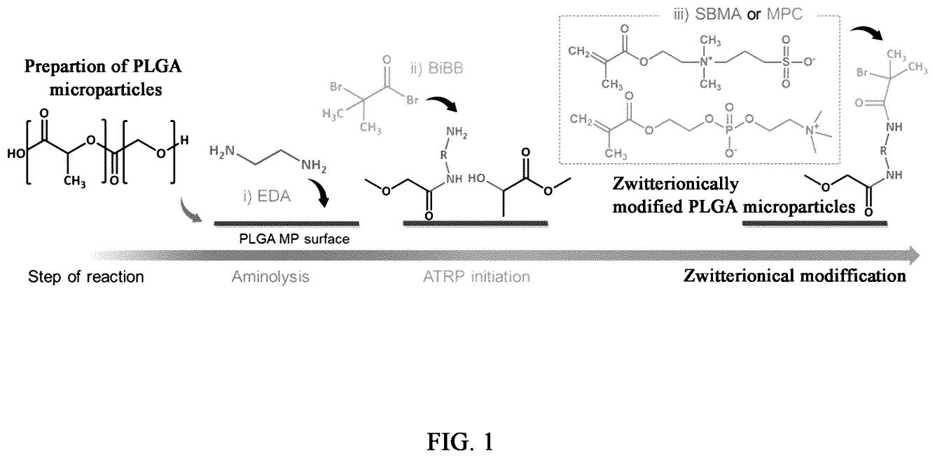

The microparticles (SBPG and MPG) having the surface of poly(lactic-co-glycolic acid) (PLGA) of the present invention modified with amphoteric ions were synthesized according to FIG. 1.

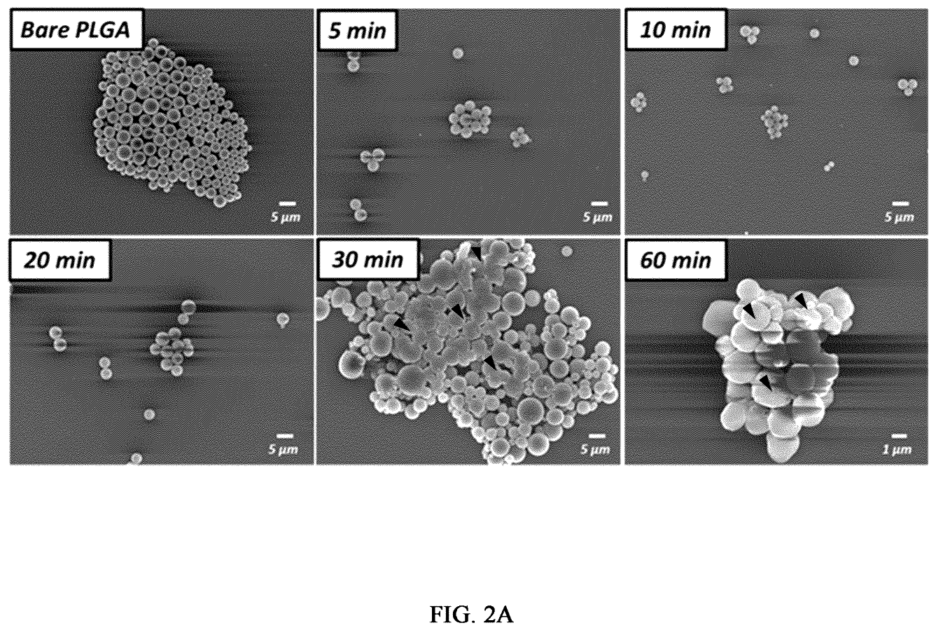

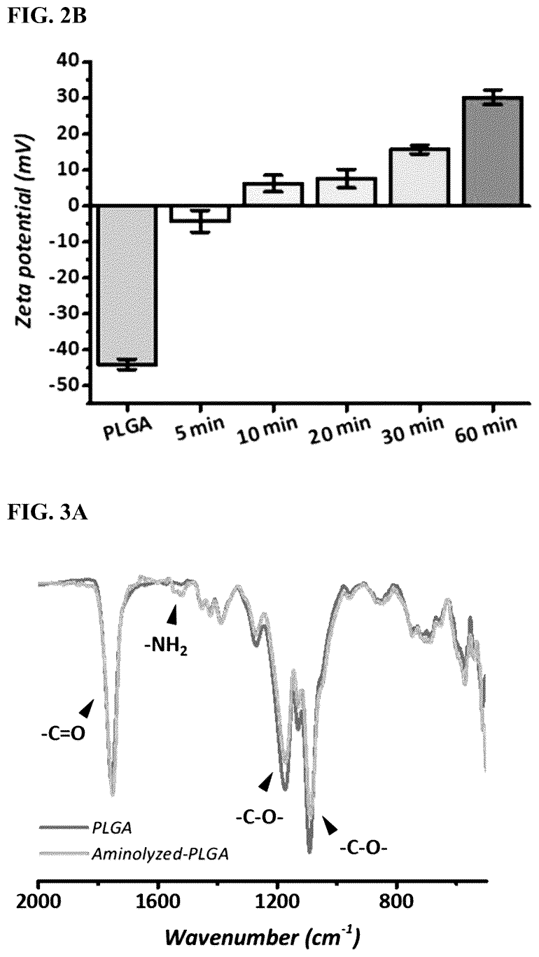

The PLGA particles prepared in Preparatory Example 1 above were subjected to an aminolysis reaction using isopropyl alcohol (IPA) as a solvent, as damage to the particles may occur if the reaction is performed in a solvent that can dissolve the PLGA particles. The aminolysis reaction is performed by dispersing PLGA particles at a concentration of 10 mg/mL in 0.5 M ethylenediamine (EDA). The particle morphology and particle surface zeta-potential were analyzed as a function of the reaction time of aminolysis (FIG. 2), and the reaction was carried out for 10 minutes to ensure that sufficient amine groups were formed on the surface with minimal damage to the particles.

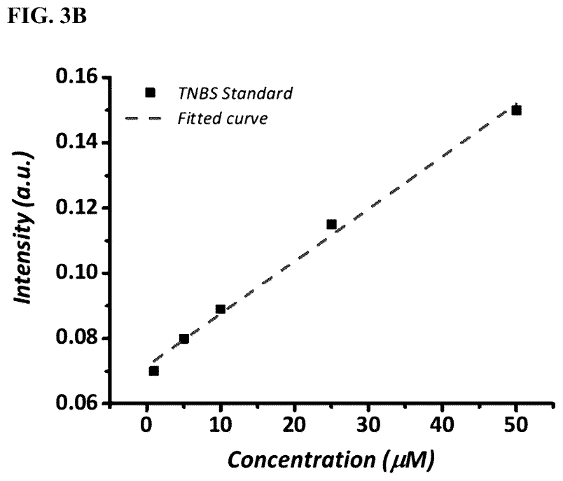

After the aminolysis reaction, the amine groups formed on the particles were identified by FTIR, and the amine groups were quantitatively analyzed by TNBS assay, and the PLGA particles with amine groups were named NH₂-PG (FIG. 3).

The obtained NH₂-PG was reacted with a-bromoisobutyryl bromide (BiBB), an ATRP reaction initiator, for the ATRP reaction. For the BiBB reaction, 10 mg/mL of NH₂-PG was dispersed in 10 mL of hexane solvent and 1 mmol of triethylamine (TEA) was added. Then, 10 μL of BiBB was added and the reaction was allowed to proceed in an ice bath for 2 hours, followed by another 4 hours at room temperature. After the reaction, the product was washed four times by centrifugation with hexane, followed by vacuum drying for 24 hours, washed once with DW, and lyophilized to obtain BiBB-functionalized PLGA particles (Br-PG).

The Br-PG obtained above was then dispersed in a solution prepared for surface zwitterionic functionalization via ATRP reaction. The solution for zwitterionic functionalization was prepared by mixing methanol and DW at 1:9 and adding 2.23 mg of CuBr₂, 3.123 mg of 2,2′-bipyridyl (bpy) and 17.612 mg of ascorbic acid (AA), respectively. Br-PG was dispersed in 10 mL of the above zwitterionic functionalization solution at a concentration of 10 mg/mL, followed by the addition of sulfobetaine methacrylate (SBMA) and 2-methacryloyloxyethyl phosphorylcholine (MPC) at a concentration of 6 mM, respectively, and the reaction was allowed to proceed for 8 hours at room temperature. After the reaction, the product was washed once with methanol and twice with DW using a centrifuge, and the residue was obtained by freeze-drying to finally obtain zwitterionic surface-modified microparticles (ZwPG).

Zwitterionic surface-modified microparticles (ZwPG) generated by reacting with SBMA are named SBPG (Example 1), and zwitterionic surface-modified microparticles (ZwPG) generated by reacting with MPC are named MPG (Example 2).

### Experimental Example 1. Characterization of PLGA Particles with Amine Groups Formed on the Surface after Aminolysis Reaction

FIG. 2A is a scanning electron microscope (SEM) image of PLGA particles as a function of the reaction time of the aminolysis reaction; FIG. 2B is a graph showing the zeta potential results of the PLGA particles functionalized with the amine groups of FIG. 2A.

Zeta Potential was performed using SZ-100, Horiba.

As shown in FIG. 2A, the aminolysis reaction rapidly induced the degradation of PLGA particles as a function of reaction time, and it was found that the particle morphology changed and aggregated after more than 30 min of aminolysis.

Furthermore, as shown in FIG. 2B, the surface charge of PLGA particles functionalized with amine groups (NH2-PG) increases with reaction time, and it is preferable to perform aminolysis for 10 minutes considering particle stability.

FIG. 3A is a graph of PLGA particles measured by Fourier transform infrared (FTIR) spectroscopy after the aminolysis reaction; FIG. 3B is a graph of the results of TNBS analysis to quantify the amine groups formed on the surface of PLGA particles after the aminolysis reaction.

As shown in FIG. 3A, Fourier transform infrared spectroscopy (FT-IR) was used to confirm the newly formed peak by the amine group, as well as a reduced C—O peak. These peak changes indicate that the aminolysis was successful.

Furthermore, the amount of amine groups was quantitatively analyzed by 2,4,6-trinitrobenzene sulfonic acid (TNBS) analysis, as shown in FIG. 3B, and 20.817 m of amine groups formed in 1 mg of NH₂-PG were identified.

### Experimental Example 2. Analysis of Microparticles with Surface Modified with Zwitterions

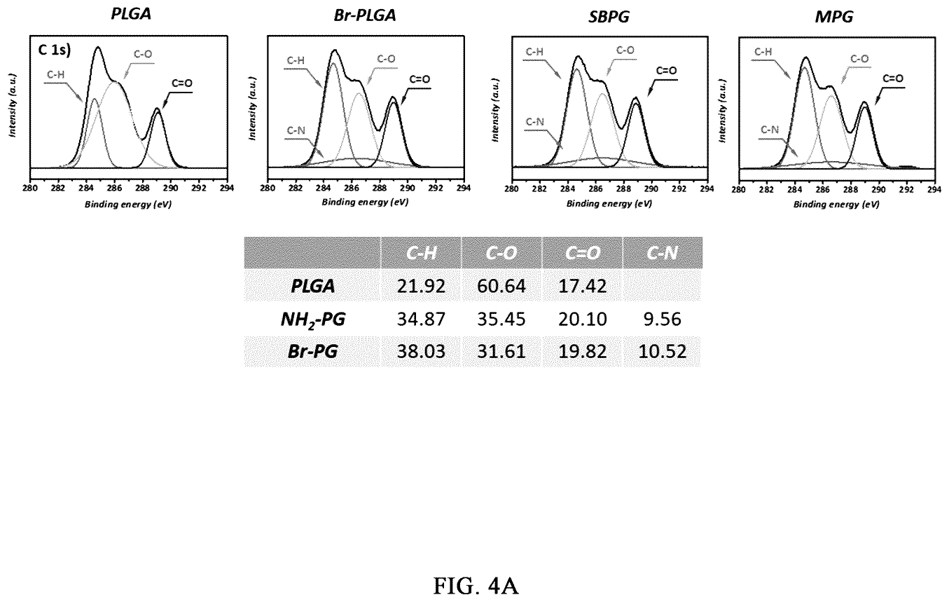

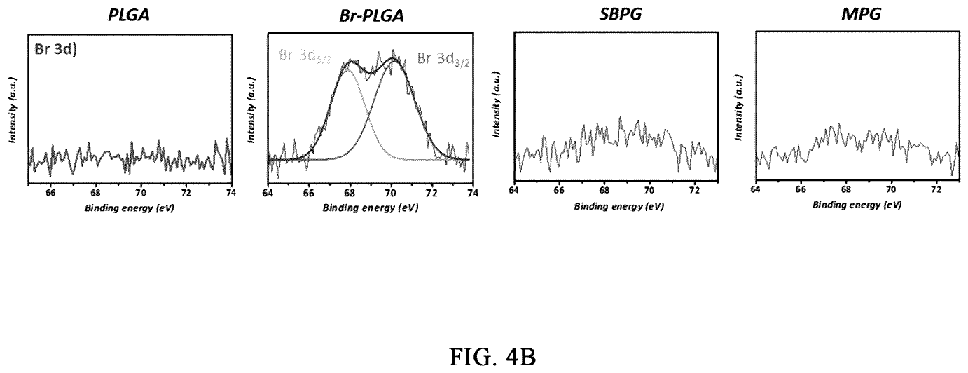

FIG. 4A is a graph showing C atom results from X-ray photoelectron spectroscopy (XPS) of PLGA particles at each stage according to one embodiment of the present invention; FIG. 4B is a graph showing Br atom results from X-ray photoelectron spectroscopy (XPS) of PLGA particles at each stage according to one embodiment of the present invention.

As shown in FIG. 4A, the aminolysis reaction resulted in an increase in the ratio of C—H groups and the formation of new C—N groups.

Furthermore, as shown in FIG. 4B, Br-PG was generated by the BiBB reaction, confirming the successful formation of ATRP reaction initiator on the surface of PLGA particles, and the ratio of Br atoms was found to decrease after the reaction of Br-PG with amphoteric compounds (amphoteric monomers).

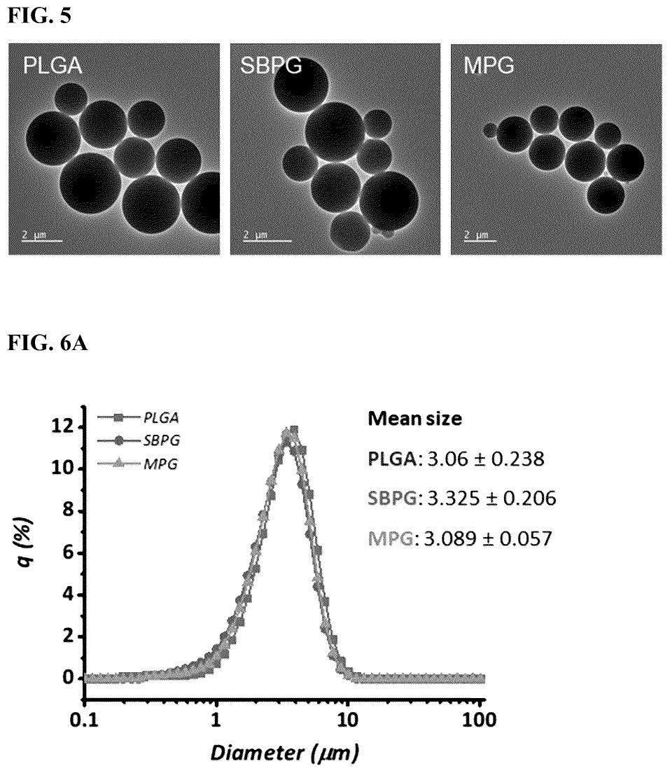

FIG. 5 shows transmission electron microscope (TEM) measurements of PLGA particles, SBPG particles, and MPG particles.

The TEM image above was acquired using NEOARM (JEM-ARM200F).

As shown in FIG. 5, the PLGA particles, SBPG particles, and MPG particles were found to exhibit perfectly spherical particles, confirming that the spherical structure and size were not altered by the synthesis process.

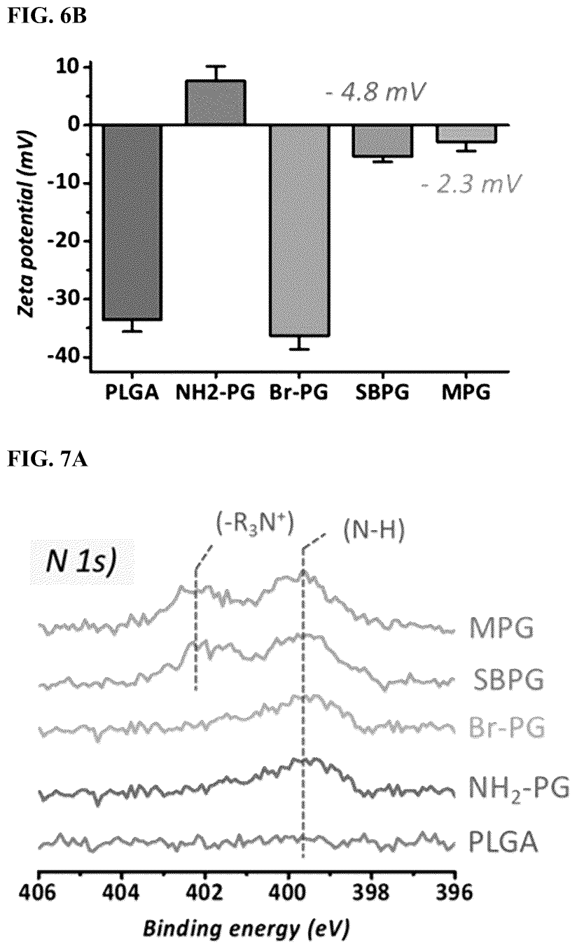

FIG. 6A is a graph of laser particle size analysis of PLGA particles, SBPG particles, and MPG particles; FIG. 6B is a graph of zeta potential results of PLGA particles, NH₂-PG particles, Br-PG particles, SBPG particles, and MPG particles.

In general, the optimal size to reach the alveolar region is particles with an average diameter of 1-5 μm, and we found that PLGA particles, SBPG particles, and MPG particles all have an average diameter of about 3 μm, as shown in FIG. 6A.

Furthermore, as shown in FIG. 6B, PLGA particles were found to have a surface charge of −34.7 mV, while the zwitterionically modified SBPG and MPG particles had surface charges close to zero at −4.8 mV and −2.3 mV, respectively.

This means that the zwitterion has been successfully functionalized on the surface of the PLGA particles.

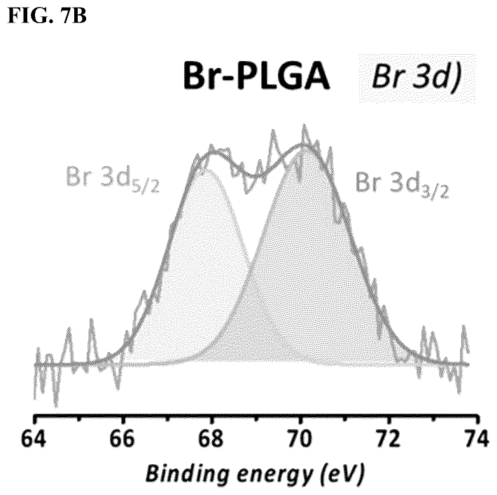

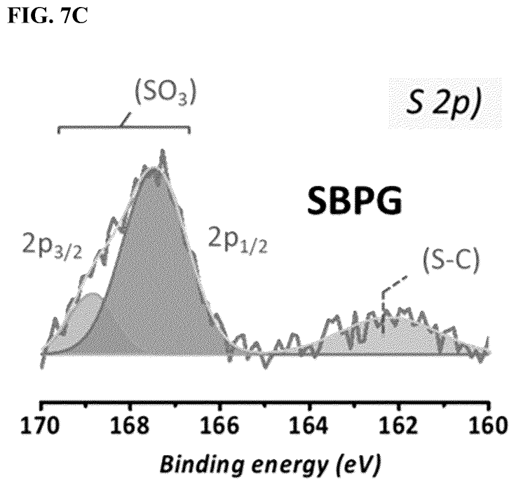

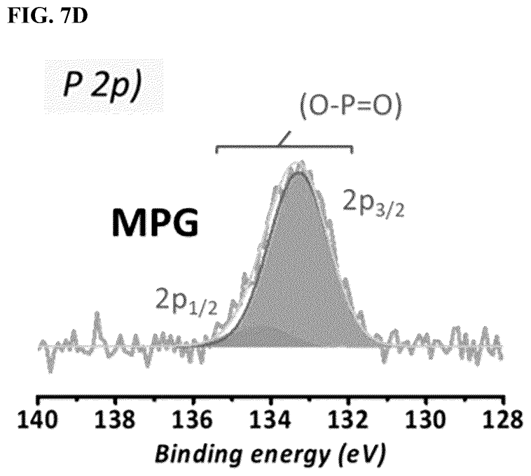

FIG. 7A is a graph of the results of X-ray photoelectron spectroscopy for PLGA particles, NH₂-PG particles, Br-PG particles, SBPG particles, and MPG particles; FIGS. 7B through 7D are graphs of the results of X-ray photoelectron spectroscopy for Br-PG particles, SBPG particles, and MPG particles, respectively.

As shown in FIGS. 7A to 7D, we confirmed the formation of the N—H group of NH₂-PG from aminolysis, and the —R₃ N⁺ peak in SBPG particles and MPG particles (FIG. 7A), and further confirmed the formation of the Br peak by reaction with BIBB during ATRP initiation (Br-PG) (FIG. 7B).

Furthermore, zwitterionic functionalization confirmed the presence of S-atom and P-atom peaks for the sulfobetaine and phosphorylcholine zwitterionic portions of SBPG particles and MPG particles, respectively (FIG. 7C, FIG. 7D).

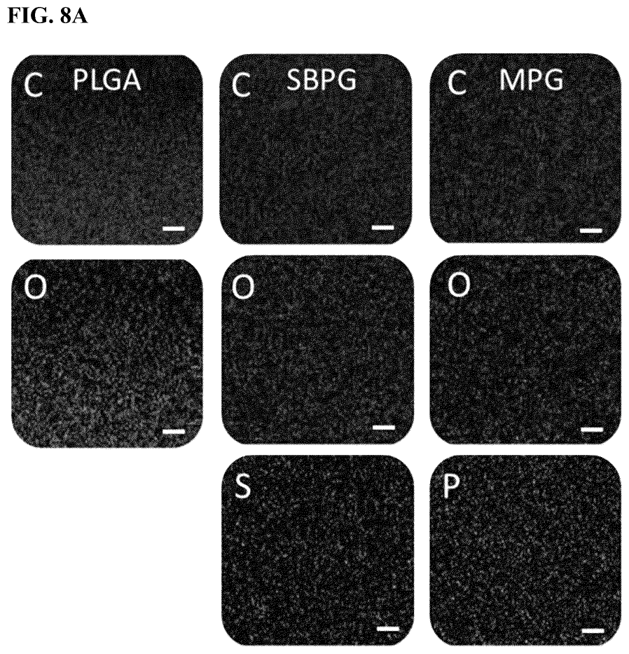

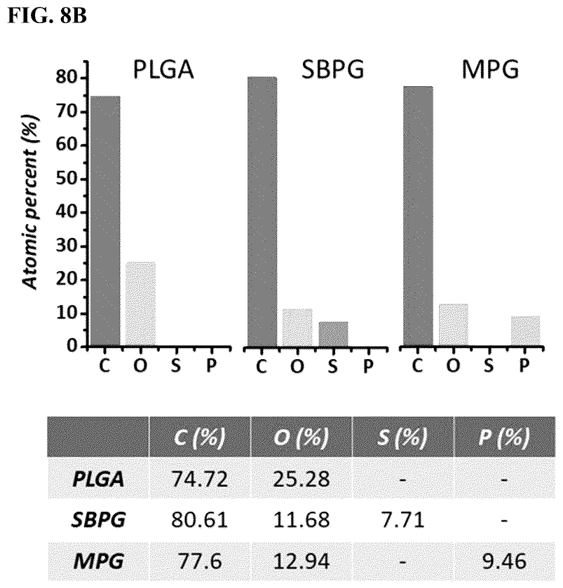

FIG. 8a is a photograph of an energy dispersive X-ray spectrometer (EDS) analysis of PLGA particles, SBPG particles, and MPG particles; FIG. 8b is a graph quantifying the results of the EDS analysis of FIG. 8A.

EDS analysis was performed using a field emission scanning electron microscope (JEOL-7800F).

As shown in FIGS. 8A and 8B, analysis of PLGA particles, SBPG particles, and MPG particles revealed that S and P atoms that were not present in PLGA particles were present in SBPG particles and MPG particles, respectively.

This means that zwitterions have been successfully formed on the surface of the PLGA particles.

### Experimental Example 3. Analysis of Mucin Adsorption Prevention and Mucin Mucus Permeability by Aqueous Membrane of Zwitterionically Functionalized Microparticles

In the case of pulmonary delivery particles, they can be removed by mucin proteins present in the airways of the lungs, resulting in a very low efficiency of the drug delivery system targeting and delivering to the alveoli. The amphoteric ions that form on the surface of the particles form a water film, which can block the adsorption of mucin as well as other proteins, and we sought to analyze whether SBPG particles and MPG particles synthesized according to an embodiment would reduce the extent of mucin adsorption in mucin mucus compared to plain PLGA particles that are not functionalized.

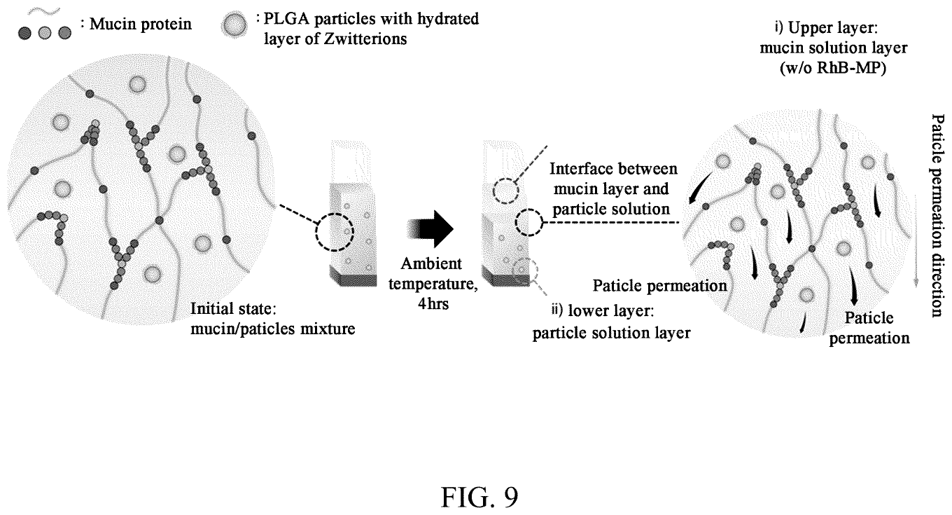

First, the following experiments were designed to analyze the particle permeability increased by zwitterions in mucin mucus of PLGA particles, SBPG particles, and MPG particles (FIG. 9).

To analyze particle permeability within mucin mucus, PLGA particles were stained with rhodamine stain. Each rhodamine-labeled PLGA particle, SBPG particle, and MPG particle was then dispersed at a concentration of 1 mg/mL in a solution that mimics the mucin environment in the body. The dispersed solution was placed in a cuvette for UV-vis measurement and the fluorescence value of rhodamine present in the particles was measured every 10 minutes for 4 hours.

The rationale for the design of the above experiment is as follows. In the case of mucin, it has a net-like structure, and typical micro-sized particles can be easily adsorbed in a net-like solution, which can greatly reduce their fluidity in mucin mucus. In the case of microparticles with a surface modified with amphoteric ions, their binding to mucin is reduced by the water film, allowing for greater fluidity, which was quantified using UV-vis.

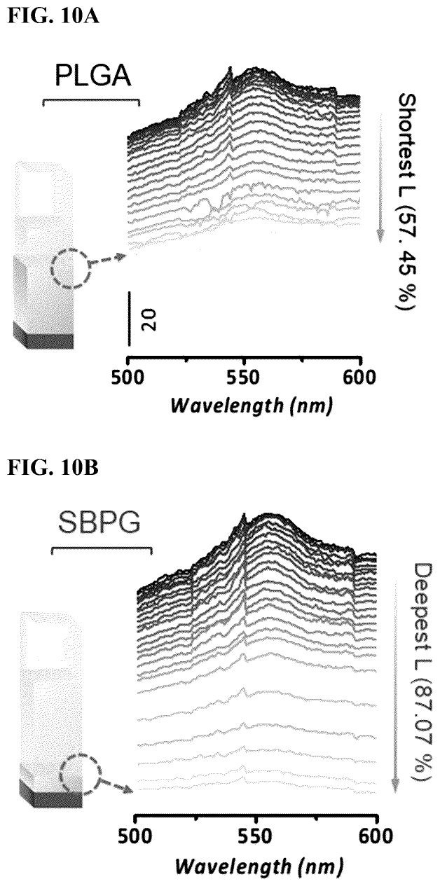

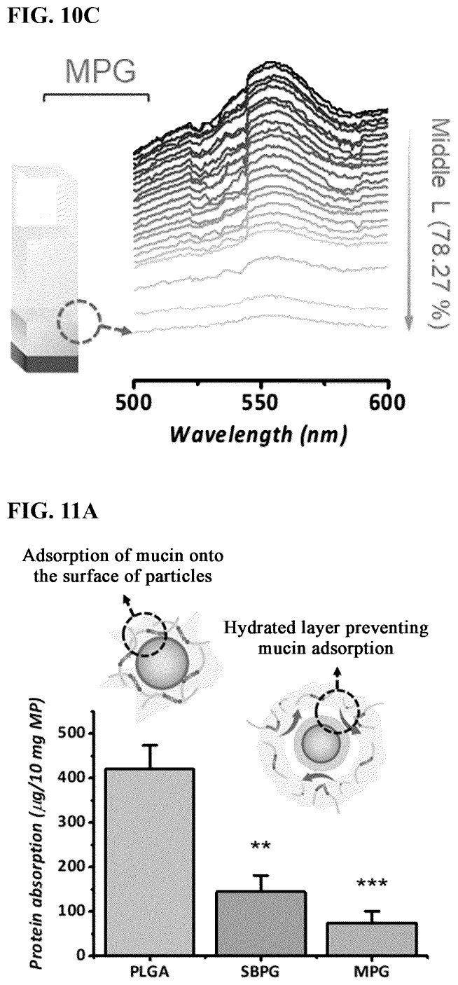

FIGS. 10A to 10C are graphs analyzing particle permeability in mucin mucus for PLGA particles, SBPG particles, and MPG particles, respectively.

As shown in FIGS. 10A to 10C, it was found that SBPG particles have about 1.5-fold and MPG particles have about 1.36-fold increased fluidity compared to PLGA particles.

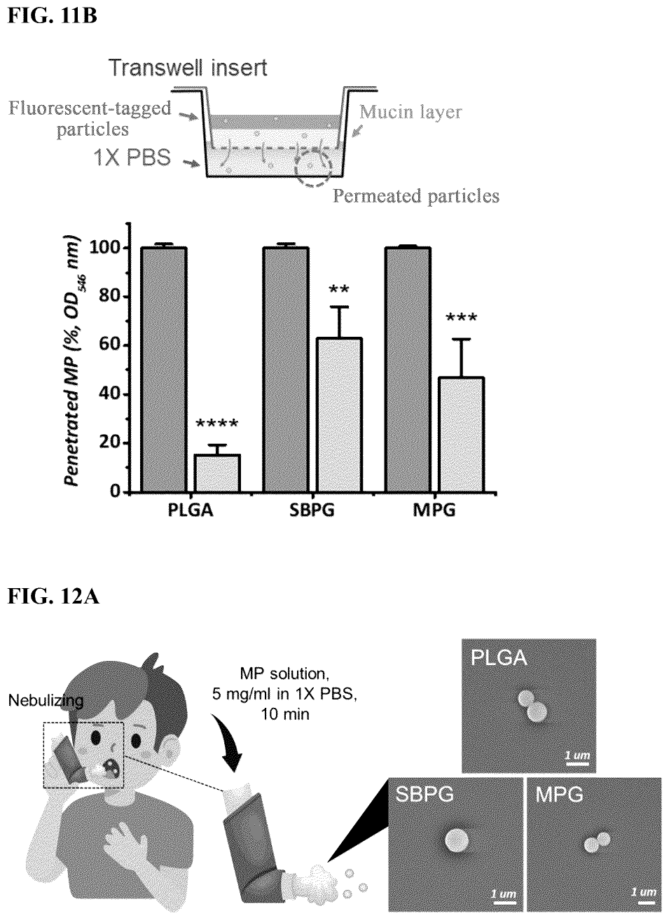

Also shown in FIG. 11A is a graph quantifying the mucin protein adsorbed on PLGA particles, SBPG particles, and MPG particles; and FIG. 11B is a graph quantifying the particles that penetrated the mucin layer on PLGA particles, SBPG particles, and MPG particles using a transwell.

As shown in FIG. 11A, 419.9 μg of mucin protein was attached to the PLGA particles, while 143.8 and 73.8 μg were attached to the SBPG and MPG particles, respectively, indicating that only about a quarter of the mucin protein was attached to the SBPG and MPG particles compared to the PLGA particles.

After dispersing the mucin mucilage in the transwells, we checked the permeability of PLGA particles, SBPG particles, and MPG particles, and found that only 15.29% of PLGA particles penetrated the mucin layer, while 62.8% and 46.6% of SBPG and MPG particles penetrated the mucin layer, respectively, as shown in FIG. 11B.

This suggests that the water membrane of microparticles whose surface is modified with amphoteric ions may reduce the interaction with mucin proteins, resulting in high anti-mucin ability and high stability in mucin mucus.

### Experimental Example 4. Stability and Cytotoxicity Assay of SBPG Particles and MPG Particles after Nebulization

For pulmonary delivery particles, a nebulizer (InnoSpire Elegance with SideStream, Philips) is the easiest route of delivery and has the advantage of not causing a foreign body sensation to the user. A typical nebulizer delivers the solution in the form of a vapor via ultrasound, and we wanted to verify that the PLGA particles, the SBPG particles of the present invention, and the MPG particles were not affected.

The experiment was performed by attaching a 50 ml tube to the outlet of the nebulizer to collect each particle, and the tube and nebulizer outlet were fixed using parafilm. After nebulization, 10 ml of 1×PBS was added to the tube containing each particle to collect the particles, centrifuged (8000 rpm), the supernatant was removed, and the nebulized particles were resuspended by adding 1 ml of pure 1×PBS for further analysis.

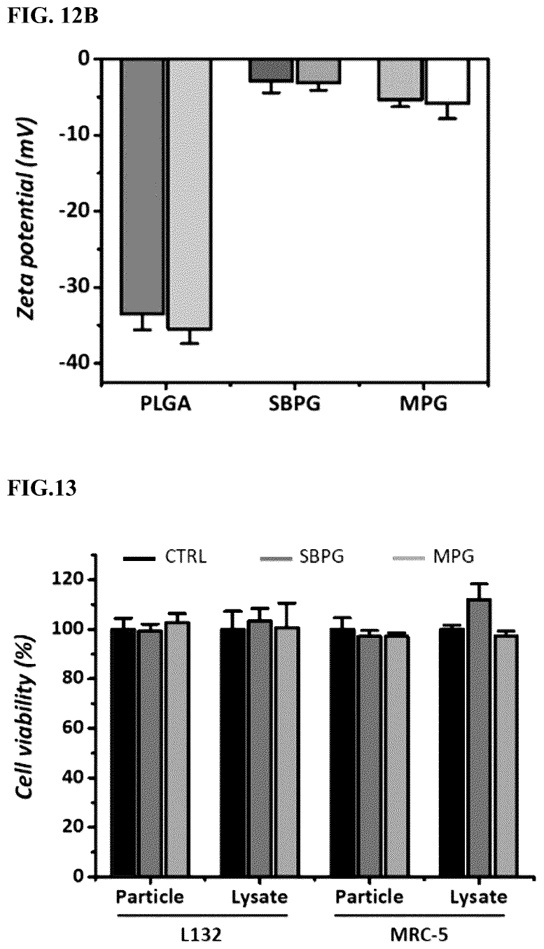

FIG. 12A is an SEM image for stability analysis of PLGA particles, SBPG particles, and MPG particles after nebulizing with a nebulizer; FIG. 12B is a graph showing the zeta potential of each particle after nebulizing with a nebulizer.

As shown in FIG. 12A and FIG. 12B, each particle obtained after nebulizing the prepared particle solution (5 mg/ml in 1×PBS) for 10 min was found to retain its shape, and the surface charge was also found to be maintained without any significant difference.

This means that the atomization stability of SBPG and MPG particles is excellent.

FIG. 13 is a graph showing the cytotoxicity of SBPG particles and MPG particles against lung tissue-derived cells.

Delivery of particles into the lungs can result in toxicity due to residues from the synthesis of the particles themselves or toxicity due to post-biodegradation products of the particles. Therefore, we analyzed the cytotoxicity of synthesized SBPG particles, MPG particles, and post-biodegradation residues on lung-derived cells (MRC-5, L132). L132 cells were cultured in Dulbecco's modified Eagle's medium (DMEM, high glucose, pyruvate) containing 10% fetal bovine serum (FBS) and 1% penicillin/streptomycin (PS) purchased from ThermoFisher Scientific; MRC-5 cells were cultured in α-Minimum Essential medium (αMEM, Thermofisher Scientific) containing the same concentration of FBS and PS as L132 medium. After cell culture, cells were inoculated into 24-well plates at the same cell density (5×10⁴ cells/well) and after 2 days of cell culture (5% CO₂, 37° C.), particle samples were administered to each well (n=3). The sample types were separated into particles of SBPG at the same concentration (1 mg/ml), particles of MPG, or their lysates (particles dissolved in 10 μl of DMSO). To test the cell viability of the above lysates, the control (CTRL) group was also given the same amount of DMSO. After incubation for 2 days, the cells were washed twice with 1×PBS, 500 μl of growth medium and 50 μl of Cell Counting Kit-8 (CCK-8) reagent (Dongin Biotech, Korea) were added and incubated for 1.5 hours. Afterward, the medium was collected and the absorbance (λ=450 nm) was measured using a plate reader.

As shown in FIG. 13, both SBPG particles and MPG particles exhibited little toxicity to lung-derived cells, confirming their high biocompatibility.

### Examples and Experimental Examples Regarding DPPG Particle

### Preparatory Example 2. Drug-Loaded PLGA Particles

Perform the same as Preparatory Example 1 above, except that in preparing the emulsion (W1/O), 15 mg of pirfenidone (hydrophobic drug) and 3 mL of dichloromethane are mixed and 75 mg of PLGA is dissolved therein, and then 30 mg of dexamethasone (hydrophilic drug) and 1 mL of tertiary distilled water are mixed and stirred under ultrasonic vibration to prepare the emulsion (W1/O), hydrophilic drug) 30 mg and 1 mL of tertiary distilled water were added in a mixture and stirred in an ultrasonicator to prepare an emulsion (W1/O), which was used to obtain 2 μm (average diameter) pirfenidone and dexamethasone (Pir/Dex) loaded PLGA particles.

### Example 3. Drug-Loaded, Zwitterionic Surface-Modified Drug Delivery System (DPPG)

The same as Example 1 above, but the Pir/Dex-loaded PLGA particles prepared in Preparatory Example 2 above were used to obtain SBMA-functionalized drug delivery systems (DPPGs).

### Experimental Example 5. Validation of Drug Entrapment of Drug-Loaded, Zwitterionic Surface-Modified Drug Delivery Systems (DPPGs)

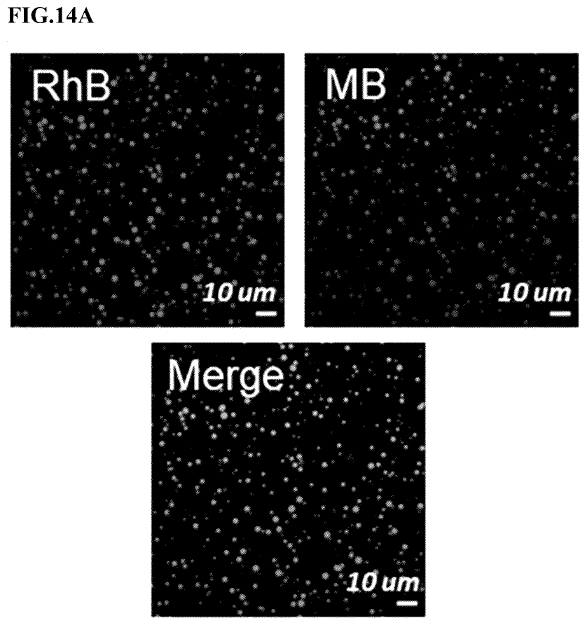

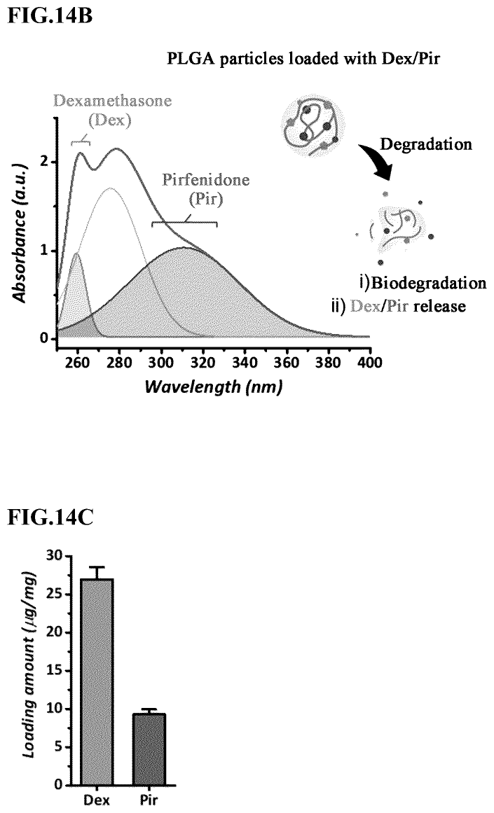

FIG. 14A is a confocal microscopy image measured after rhodamine (RhB) and methylene blue (MB) were immersed in PLGA particles; FIG. 14B is a graph of UV-vis measurements of a zwitterionic surface-modified drug delivery system (DPPG) loaded with Dex and Pir drugs; and FIG. 14C is a graph quantifying the measurements in FIG. 14B.

As shown in Preparatory Example 1, PLGA particles can be synthesized through a double emulsion method using water and an organic solvent to synthesize PLGA particles, and if two or more drugs are immersed in each solvent during the process, the final PLGA particles can contain two or more drugs inside. In this study, PLGA particles were synthesized by adding rhodamine to an organic solvent and methylene blue to water.

Confocal microscopy confirmed the simultaneous presence of rhodamine and methylene blue within the PLGA particles, as shown in FIG. 14A.

Thus, PLGA particles comprising dexamethasone (Dex) and pirfenidone (Pir) were prepared as shown in Preparatory Example 2. Specifically, since Pir has side effects such as dizziness and gastrointestinal irritation, and Dex is a steroidal agent that can cause serious problems with the endocrine system at high concentrations, optimizing the minimum dosage of Pir and Dex to maximize the effect is important for drug administration. Therefore, PLGA particles containing dexamethasone (Dex) and pirfenidone (Pir) were prepared and functionalized with zwitterions to maximize the therapeutic effect by administering the minimum concentration of Pir and Dex simultaneously.

To determine whether Dex and Pir are present in the interior of the Dex and Pir drug-loaded zwitterionic functionalized drug delivery system (DPPG) prepared according to Example 3 above, the Dex and Pir drug-loaded zwitterionic functionalized drug delivery system (DPPG) was dissolved in DMSO solvent, and the peaks of Dex and Pir were identified by UV-vis and quantitative analysis was performed.

UV-vis analysis was performed using a model Evolution 300 (ThermoFisher Scientific).

Accordingly, Dex and Pir were found to be loaded inside the DPPG particles at 26.95 ug/mg and 9.28 ug/mg, respectively, as shown in FIG. 14B and FIG. 14C, confirming that zwitterionic functionalized drug delivery systems (DPPGs) can be utilized to deliver two or more drugs into the lungs with high efficiency.

What is claimed is:

1. Microparticles comprising poly(lactic-co-glycolic acid) (PLGA), wherein the surface of the PLGA is modified with zwitterions.

2. The microparticle of claim 1, wherein said microparticles have an average diameter of 1 to 4 μm.

3. The microparticles of claim 1, wherein said zwitterion is formed using sulfobetaine methacrylate (SBMA), 2-methacryloyloxyethyl phosphorylcholine (MPC), or carboxybetaine methacrylate (CBMA).

4. A drug delivery system comprising the microparticles of claim 1 and a drug loaded on or in the microparticles.

5. The drug delivery system of claim 4, wherein the target site of said drug delivery system is the lungs.

6. The drug delivery system of claim 4, which reaches the lungs via intranasal administration or endobronchial administration.

7. The drug delivery system of claim 4, which is loaded with both a hydrophobic drug and a hydrophilic drug.

8. The drug delivery system of claim 7, wherein said hydrophobic drug is pirfenidone or nintedanib;
and wherein said hydrophilic drug is dexamethasone, prednisone (PD), prednisolone (PDS), or methylprednisolone (MP).

9. A method for preparing microparticles, comprising:
(A) performing an aminolysis reaction to form amine groups on the surface of poly(lactic-co-glycolic acid) (PLGA) particles;
(B) reacting the PLGA particles having amine groups on the surface with an atom transfer radical polymerization (ATRP) initiator to form halogen groups; and
(C) reacting the halogen groups with a zwitterionic compound to obtain PLGA microparticles with zwitterionically modified surfaces.

10. The method for preparing microparticles of claim 9, wherein in the step (A), the PLGA particles are prepared by:
(a) dissolving PLGA in an organic solvent, adding distilled water thereto, and then treating the mixture with an ultrasonic wave generator to prepare an emulsion;
(b) adding the emulsion dropwise to polyvinyl alcohol to form a double emulsion; and
(c) stirring the double emulsion to obtain solid PLGA particles.

11. The method for preparing microparticles of claim 9, wherein in the step (C), the zwitterionic compound is sulfobetaine methacrylate (SBMA), 2-methacryloyloxyethyl phosphorylcholine (MPC), or carboxybetaine methacrylate (CBMA).

12. A method for preparing a drug delivery system, comprising:
(A′) performing an aminolysis reaction to form amine groups on the surface of poly(lactic-co-glycolic acid) (PLGA) particles loaded with two kinds of drugs;
(B′) reacting the PLGA particles having amine groups on the surface with an atom transfer radical polymerization (ATRP) initiator to form halogen groups; and
(C′) reacting the halogen groups with a zwitterionic compound to obtain a drug delivery system with zwitterionically modified surfaces.

13. The method for preparing a drug delivery system of claim 12, wherein in the step (A′), the PLGA particles loaded with two kinds of drugs are prepared by:
(a′) dissolving PLGA in an organic solvent in which a hydrophobic drug is dissolved, adding distilled water in which a hydrophilic drug is dissolved, and then treating the mixture with an ultrasonic wave generator to prepare an emulsion;
(b′) adding the emulsion dropwise to polyvinyl alcohol to form a double emulsion; and
(c′) stirring the double emulsion to obtain solid PLGA particles loaded with two kinds of drugs.

14. The method for preparing a drug delivery system of claim 13, wherein the hydrophobic drug is pirfenidone or nintedanib; and the hydrophilic drug is dexamethasone, prednisone (PD), prednisolone (PDS), or methylprednisolone (MP).

15. The method for preparing a drug delivery system of claim 12, wherein in the step (C′), the zwitterionic compound is sulfobetaine methacrylate (SBMA), 2-methacryloyloxyethyl phosphorylcholine (MPC), or carboxybetaine methacrylate (CBMA).

16. A pharmaceutical composition for treating pulmonary disease, comprising the drug delivery system of claim 4 as an active ingredient.

17. The pharmaceutical composition for treating pulmonary disease of claim 16, wherein the pharmaceutical composition is for intranasal administration or intrabronchial administration.

18. The pharmaceutical composition for treating pulmonary disease of claim 16, wherein the pulmonary disease is one or more selected from the group consisting of idiopathic pulmonary fibrosis (IPF), acute lung injury, bleomycin-induced pulmonary fibrosis, ventilator-induced lung injury, chronic obstructive pulmonary disease (COPD), chronic bronchitis, and emphysema.
